(19) United States
(12) Patent Application Publication
(10) Pub. No.: US 2024/0208880 A1
(43) Pub. Date: Jun. 27, 2024

(54) REACTION APPARATUS

(71) Applicant: INDUSTRIAL TECHNOLOGY RESEARCH INSTITUTE, Hsinchu (TW)

(72) Inventors: Shih-Chi LEE, Pingtung City (TW); Chuan-Chi CHIEN, Kaohsiung City (TW); Chun-Shen CHENG, Kaohsiung City (TW); Liang-Rung CHANG, Xingang Township (TW); Shu-Hsien TSAI, Kaohsiung City (TW); JenHao CHENG, Taichung City (TW); MENG-KO CHANG, Puxin Township (TW)

(73) Assignee: INDUSTRIAL TECHNOLOGY RESEARCH INSTITUTE, Hsinchu (TW)

(21) Appl. No.: 18/087,260

(22) Filed: Dec. 22, 2022

(51) Int. Cl. C05F 17/979; C05F 17/40; C05F 17/60; C05F 17/993; C12M 1/00

(52) U.S. Cl. CPC C05F 17/979; C05F 17/40; C05F 17/60; C05F 17/993; C12M 23/58; C12M 29/08

(57) ABSTRACT

A reaction apparatus is provided. The reaction apparatus includes a first tank, a second tank, a cover, a stirring assembly, a heater, a controller and a gas delivery system. The first tank has an accommodating space. The second tank is in the accommodating space of the first tank and includes holes. The cover is on an opening of the first tank. The stirring assembly is on the cover. The stirring assembly includes a driving motor and a stirrer connected to the driving motor. The stirrer is in the accommodating space of the first tank. The heater is in the accommodating space of the first tank. The controller is coupled to the heater to control the heater. The gas delivery system is on the first tank and in communication with the accommodating space of the first tank.

### BACKGROUND

### Technical Field

The present disclosure relates to a reaction apparatus, and more particular to a reaction apparatus including a one-pot reaction device.

### Description of the Related Art

As people pay more and more attention to the problems of climate change and global warming issues, the development trend in the agriculture has been toward concepts of environmental protection, friendly farming, sustainable utilization, organic species, etc. Under these trends, the use of agricultural bio-based composite materials such as microbial-based composite materials to replace traditional chemical fertilizers has become one of the key points of industrial development. However, the manufacturing process of agricultural bio-based composite materials is complicated and mass production of agricultural bio-based composite materials is difficult.

### SUMMARY

The present disclosure relates to a reaction apparatus that can be used to produce various agricultural bio-based composite materials. The reaction apparatus can be used to produce various agricultural bio-based composite materials through a one-pot reaction process. The reaction apparatus can effectively simplify the manufacturing process and is suitable for mass production.

According to an embodiment of the present disclosure, a reaction apparatus is provided. The reaction apparatus includes a first tank, a second tank, a cover, a stirring assembly, a heater, a controller and a gas delivery system. The first tank has an accommodating space. The second tank is in the accommodating space of the first tank and includes holes. The cover is on an opening of the first tank. The stirring assembly is on the cover. The stirring assembly includes a driving motor and a stirrer connected to the driving motor. The stirrer is in the accommodating space of the first tank. The heater is in the accommodating space of the first tank. The controller is coupled to the heater to control the heater. The gas delivery system is on the first tank and in communication with the accommodating space of the first tank.

The above and other embodiments of the disclosure will become better understood with regard to the following detailed description of the non-limiting embodiment(s). The following description is made with reference to the accompanying drawings.

### BRIEF DESCRIPTION OF THE DRAWINGS

FIG. 1 illustrates a schematic view of a reaction apparatus according to an embodiment of the present disclosure.

FIG. 2 illustrates a partial perspective view of a reaction device according to an embodiment of the present disclosure.

FIG. 3 illustrates a schematic view of a second tank according to an embodiment of the present disclosure.

FIG. 4 illustrates a partial schematic view of a cover according to an embodiment of the present disclosure.

FIG. 5 illustrates a schematic view of a buckle according to an embodiment of the present disclosure.

FIG. 6 illustrates a partial schematic view of a reaction device according to an embodiment of the present disclosure.

FIG. 7 illustrates a schematic view of an air cooling recycle system according to an embodiment of the present disclosure.

FIG. 8 illustrates a schematic view of an operation of a reaction apparatus according to an embodiment of the present disclosure.

FIG. 9 illustrates a schematic view of an operation of a reaction apparatus according to an embodiment of the present disclosure.

FIG. 10 illustrates a schematic view of a second tank according to another embodiment of the present disclosure.

### DETAILED DESCRIPTION

Various embodiments will be described more fully hereinafter with reference to accompanying drawings, which are provided for illustrative and explaining purposes rather than a limiting purpose. For clarity, the elements may not be drawn to scale. In addition, some elements and/or reference numerals may be omitted from some drawings. It is contemplated that the elements and features of one embodiment can be beneficially incorporated in another embodiment without further recitation.

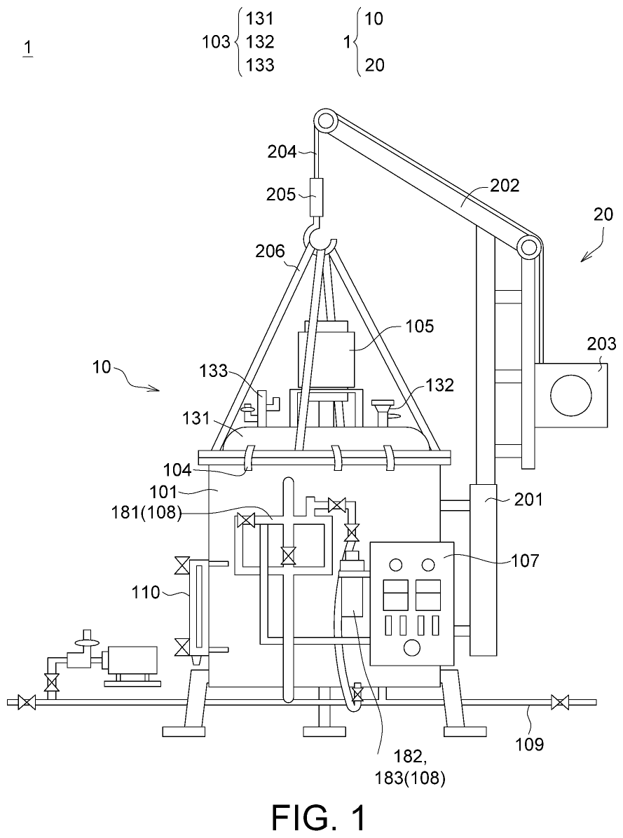

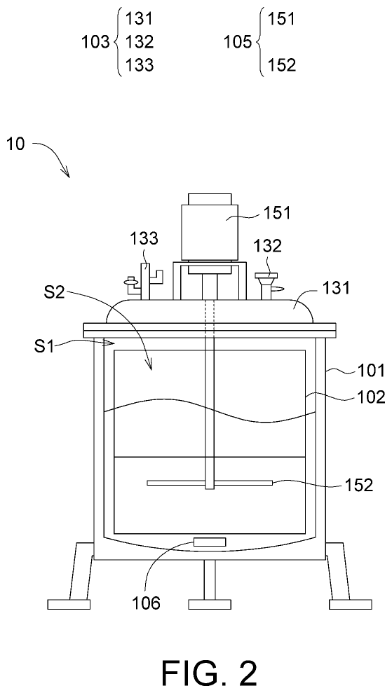

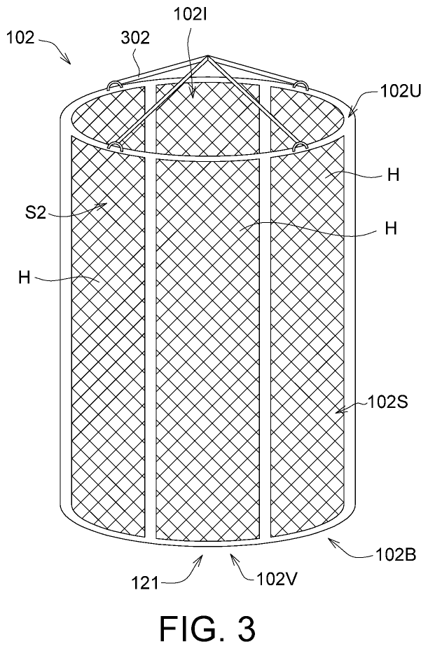

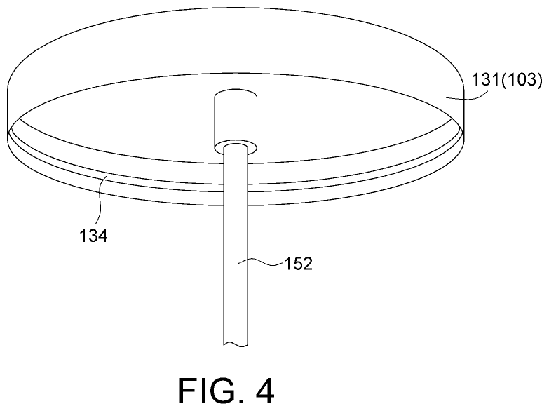

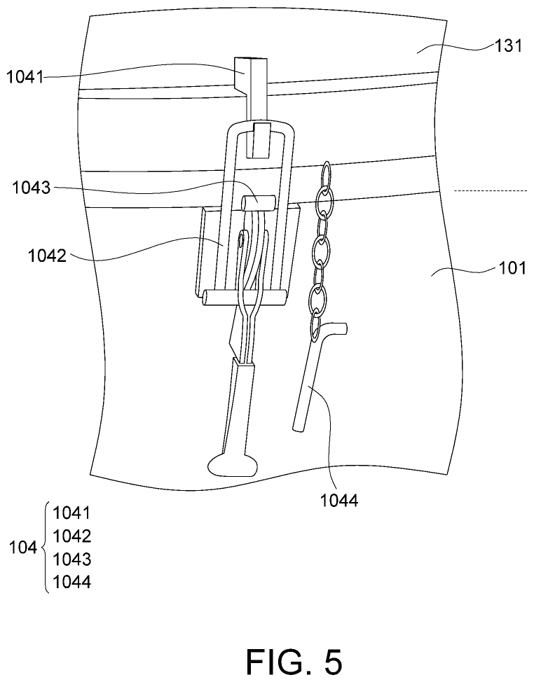

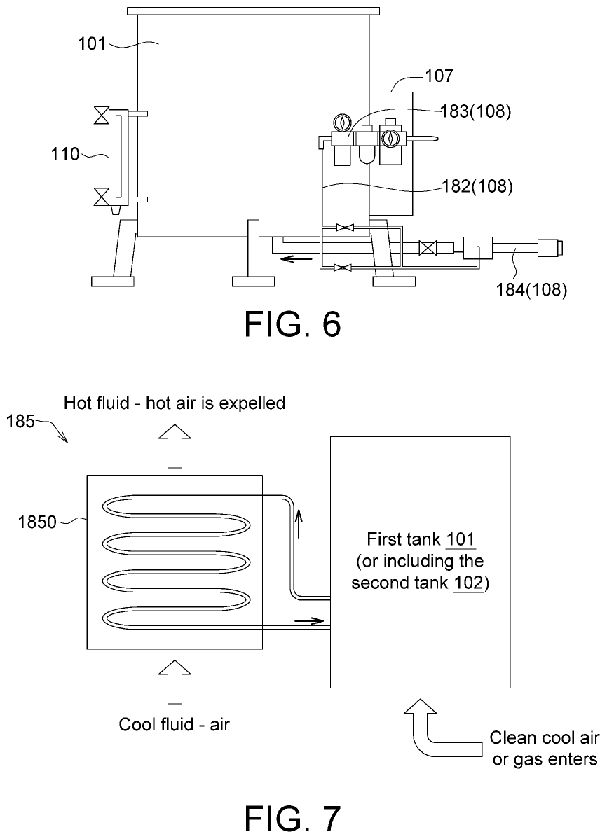

Referring to FIGS. 1-6. FIG. 1 illustrates a schematic view of a reaction apparatus 1 according to an embodiment. FIG. 2 illustrates a partial perspective view of a reaction device 10 according to an embodiment. FIG. 3 illustrates a schematic view of a second tank 102 according to an embodiment. FIG. 4 illustrates a partial schematic view of a cover 131 according to an embodiment. FIG. 5 illustrates a schematic view of a buckle 104 according to an embodiment. FIG. 6 illustrates a partial schematic view of the reaction device 10 of FIG. 1. FIG. 6 and FIG. 1 are illustrated under different viewpoints. Some elements in the reaction device 10 are omitted in FIG. 2 and FIG. 6 for clarity of discussion. The reaction apparatus 1 includes a reaction device 10. The reaction device 10 includes a first tank 101, a second tank 102, a lid assembly 103, at least one buckle 104, a stirring assembly 105, a heater 106, a controller 107, a gas delivery system 108, a pumping device 109 and a sampling pipe 110. The reaction device 10 may be a one-pot reaction device, that is, a one pot one-pot reaction process can be performed in the reaction device 10.

The first tank 101 may be a hollow container with an accommodating space S1 inside. The upper part of the first tank 101 has an opening, and the lower part of the first tank 101 (or bottom of the first tank 101) is closed. The accommodating space S1 of the first tank 101 can be used to hold liquid materials (such as water, microbial nutrient fluid, bacteria liquid, etc.), sold materials, etc. The volume of the accommodating space S1 of the first tank 101 may be between about 450 L and about 500 L or may be greater than 500 L; the present disclosure is not limited thereto. The first tank 101 may have any shape such as a cylinder shape, a square cylinder shape, etc. The first tank 101 can be made of stainless steel or other heat-resistant, acid-resistant and alkali-resistant materials. For example, the first tank 101 can be made of SUS304 or SUS316.

The second tank 102 removably disposed in the accommodating space S1 of the first tank 101. In an embodiment, the second tank 102 may be suspended in the accommodating space S1 of the first tank 101. In an embodiment, the bottom of the first tank 101 may has a cone shape, and the second tank 102 can be supported on the bottom of the first tank 101 without being completely attached to the bottom of the first tank 101. The second tank 102 may be a hollow container with an accommodating space S2 inside. As shown in FIG. 3, the second tank includes holes H passing through the bottom 102B of the second tank 102 and/or the sidewall 102S of the second tank 102 and/or the upper surface 102U of the second tank 102. The accommodating space S2 of the second tank 102 is in communication with the accommodating space S1 of the first tank 101. The second tank 102 includes a supply inlet 102I on the upper surface 102U. The solid materials (such as biochar) used in the reaction process can enter the accommodating space S2 of the second tank 102 from the supply inlet 102I. In the product storage and packaging stage after the reaction is completed, the solid product can be removed from the second tank 102 through the supply inlet 102I.

The solid materials in the accommodating space S2 of the second tank 102 may be immersed in the liquid materials in the accommodating space S1 of the second tank 101 since the second tank 102 includes holes H. A diameter of the hole H of the second tank 102 may be any value, as long as the diameter of the hole H is sufficient to block the solid materials in the second tank 102. In an embodiment, the second tank 102 may be a sieve meeting ASTM (American Society for Testing and Materials) E-11 specifications for the number 70 sieve to the number 300 sieve or may be a sieve meeting ASTM E-11 specifications for the sieve with a number greater than 300. The second tank 102 can be made of stainless steel or other heat-resistant, acid-resistant and alkali-resistant materials. For example, the second tank 102 can be made of SUS304 or SUS316. The second tank 102 may have any shape such as a cylinder shape, a square cylinder shape, etc.

In this embodiment, as shown in FIG. 3, the second tank 102 may include an outlet 102V and an outlet door 121 at the bottom 102B. The outlet door 121 is movable between a release position and a closed position. When the outlet door 121 is in the release position, the outlet 102V is open and not covered by the outlet door 121; the solid products in the second tank 102 can be removed from the second tank 102 through the outlet 102V, which can simplify the collection of products. When the outlet door 121 is in the closed position, the outlet door 121 covers the outlet 102V and the outlet 102V is closed. The outlet door 121 may be disposed at the bottom 102B through a slide rail mechanism, a pivot mechanism, a hinge mechanism, and the like. The outlet door 121 may be fastened to the bottom 102B in any manner, as long as it does not affect the movement of the second tank 102. In other embodiments, the second tank 102 may not include the outlet 102V and the outlet door 121 disposed at the bottom 102B.

In an embodiment, the second tank 102 may include two V-shaped handles 302. The V-shaped handles 302 may be disposed symmetrically on the upper surface 102U of the second tank 102 or at the edge of the supply inlet 102I. In an embodiment, the second tank 102 may include one or more laminates (not shown) disposed inside. The laminate may have a plurality of through holes extending from an upper surface of the laminate to a lower surface of the laminate. The diameter of the through hole may be smaller than the width or diameter of the solid material in the second tank 102.

The lid assembly 103 is on the first tank 101 and the second tank 102. The lid assembly 103 includes a cover 131, an inlet 132 and a relief valve 133. The cover 131 is on the opening of the first tank 101. The cover 131 covers the opening of the first tank 101. The cover 131 can be made of stainless steel or other heat-resistant, acid-resistant and alkali-resistant materials. For example, the first tank 101 can be made of SUS304 or SUS316. The inlet 132 is on the cover 131 and passes through the cover 131. The inlet 132 is in communication with the accommodating space S1 of the first tank 101. The materials enter the first tank 101 and/or the second tank 102 through the inlet 132. During the reaction process, the materials (such as bacteria, etc.) can be added through the inlet 132 without separating the cover 131 from the first tank 101, which can reduce the pollution of the reaction process and simplify the manufacturing process. The relief valve 133 is on the cover 131 and passes through the cover 131. The relief valve 133 is in communication with the accommodating space S1 of the first tank 101. During the reaction process, the gas inside the first tank 101 may be discharged through the relief valve 133 so as to control the pressure in the first tank 101. The relief valve 133 can be opened or closed as required.

Referring to FIGS. 2 and 4. FIG. 4 illustrates a partial schematic view of a cover 131 according to an embodiment. Some elements on the cover 131, such as the inlet 132 and the relief valve 133, are omitted in FIG. 4 for clarity of discussion. In an embodiment, the lid assembly 103 may include a gasket 134 at the edge of the cover 131. The gasket 134 may be between the cover 131 and the first tank 101. For example, the gasket 134 may be at the edge of the lower surface of the cover 131. The gasket 134 can improve the sealability between the cover 131 and the first tank 101. For example, the gasket 134 is a heat-resistant silicone sealing strip.

At least one buckle 104 may be arranged between the cover 131 and the first tank 101. The buckle 104 has a fasten state and a non-fasten state. When the buckle 104 is in the fasten state, the buckle 104 is connected between the cover 131 and the first tank 101, and the buckle 104 exerts a force on the cover 131 to push the cover 131 to the first tank 101 and fasten the cover 131 on the first tank 101. Alternatively, when the buckle 104 is in the fasten state, the buckle 104 exerts a force on the first tank 101 to push the first tank 101 to the cover 131 and fasten the cover 131 on the first tank 101. When the buckle 104 is in the non-fasten state, the buckle 104 does not exert a force on the cover 131 and/or the first tank 101. The sealability between the cover 131 and the first tank 101 can be improved and the safety under high temperature and high pressure operation can be enhanced by the use of the buckle 104. In an embodiment, the buckle 104 may include a clamp, a snap latch, a toggle latch, etc. FIG. 5 illustrates one of the buckles 104 that can be used in the present disclosure; the present disclosure is not limited thereto. As shown in FIG. 5, the buckle 104 includes a fixed part 1041 on the cover 131, a movable part 1042 pivotally connected to the first tank 101, an inserting hole 1043 on the first tank 101, and a rod 1044 on the cover 131. The movable part 1042 may pivot with respect to the first tank 101 to be fastened to the fixed part 1041 or loosened from the fixed part 1041. When the movable part 1042 is fastened to the fixed part 1041, the buckle 104 is in the fasten state. When the movable part 1042 is loosened from the fixed part 1041, the buckle 104 is in the non-fasten state. The shape of the rod 1044 may correspond to the shape of the inserting hole 1043. The size of the rod 1044 may be slightly smaller than the size of the inserting hole 1043 so that the rod 1044 can pass through the inserting hole 1043. When the rod 1044 passes through the inserting hole 1043, the movement of the rod 1044 in at least one direction is limited by the inserting hole 1043. For example, the movement of the rod 1044 in the normal direction of the surface of the cover 131 is limited by the inserting hole 1043. In this embodiment, when the rod 1044 passes through the inserting hole 1043, the movable part 1042 is fastened to the fixed part 1041 and the movable part 1042 is between the rod 1044 and the cover 131 and/or between the rod 1044 and the first tank 101. The fastening stability of the fixed part 1041 and the movable part 1042 can be improved, and the movable part 1042 can be prevented from getting loose from the fixed part 1041 by the use of the rod 1044 and the inserting hole 1043.

The stirring assembly 105 is on the cover 131. The stirring assembly 105 includes a driving motor 151 and a stirrer 152 connected to the driving motor 151. The driving motor 151 may be used to drive the stirrer 152. The stirrer 152 is in the accommodating space S1 of the first tank 101. In this embodiment, as shown in FIG. 2, the stirrer 152 is in the accommodating space S1 of the first tank 101 and in the accommodating space S2 of the second tank 102. The stirrer 152 may be any type of stirrer, such as a helical stirrer, bladed stirrer, paddle stirrer, turbine stirrer, etc., as long as it can cause the materials to move. With the use of the stirrer 152, various materials can be mixed evenly, the reaction can take place evenly, and the quality of the product can be improved. In an embodiment, the stirrer 152 is removably connected to the driving motor 151 for easy cleaning.

The heater 106 is in the accommodating space S1 of the first tank 101. The heater 106 can be used to adjust the temperature inside the first tank 101. The heater 106 can maintain the inside of the first tank 101 at a temperature suitable for the reaction. In addition, the heater 106 can be used to perform a sterilization process to the materials under high temperature and high pressure so as to reduce pollution. The controller 107 is disposed on the outside of the first tank 101. The controller 107 is coupled to the heater 106 to control the heater 106. The controller 107 may include a temperature controller and a time controller. The temperature controller is used to control the temperature of the heater 106. The time controller is used to control the heating time of the heater 106.

The gas delivery system 108 is on the first tank 101 and passes through the first tank 101. The gas delivery system 108 is in communication with the accommodating space S1 of the first tank 101. The gas delivery system 108 may include an exhaust conduit 181 and an intake conduit 182. The gas inside the first tank 101 may be discharged through the exhaust conduit 181 so as to control the pressure in the first tank 101. One or more valves can be arranged on the exhaust conduit 181 to control the opening or closing of the exhaust conduit 181. As shown in FIG. 6, gas may enter the first tank 101 through the intake conduit 182. One or more valves can be arranged on the intake conduit 182 to control the opening or closing of the intake conduit 182, which helps to adjust the pressure. For example, gas entering the first tank 101 through the intake conduit 182 may be air, nitrogen, gas mixture without oxygen, etc. In an embodiment, when the reaction device of the present disclosure is used to culture microbes, the type of gas introduced into the first tank 101 can be changed according to the reproduction conditions of the microbes, so as to promote the reproduction of microbes. In an embodiment, the gas delivery system 108 may include a filtration assembly 183. The filtration assembly 183 can be used to filter the gas to ensure that the gas introduced into the first tank 101 is clean, which can reduce the pollution during the reaction process.

In an embodiment, as shown in FIG. 6, the gas delivery system 108 includes a cooling device 184 connected to the intake conduit 182. The cooling device 184 can be used to lower the temperature of gas introduced into the first tank 101. The cooling device 184 can maintain the inside of the first tank 101 at a temperature suitable for the reaction. In addition, the cooling device 184 can accelerate the cooling of the materials in the first tank 101 after the sterilization process under high temperature and high pressure. In an embodiment, the cooling device 184 is a vortex cooler. Referring to FIGS. 6-7, in an embodiment, the gas delivery system 108 includes an air cooling recycle system 185. In the process of cooling the inside of the first tank 101 (or including the second tank 102) by introducing cold gas into the first tank 101 (or including the second tank 102) through the cooling device 184 and the intake conduit 182, heat in the first tank 101 is conducted away from the first tank 101 and transferred to the cold gas, heat from this warm gas is transferred to a condenser 1850 of the air cooling recycle system 185, and the water vapor generated during the heat exchange can be recovered and reused by the air cooling recycle system 185.

The pumping device 109 is on the first tank 101 and passes through the first tank 101. The pumping device 109 is in communication with the accommodating space S1 of the first tank 101. The pumping device 109 can be used to cause liquid in the first tank 101 to move from the first tank 101 during the reaction process or after the reaction is completed. The sampling pipe 110 is on the first tank 101 and passes through the first tank 101. The sampling pipe 110 is in communication with the accommodating space S1 of the first tank 101. The sampling pipe 110 can be used to extract the liquid sample inside the first tank 101 during the reaction process, and the extent to which the reaction has proceeded can be known by analyzing the liquid sample and/or observing the appearance of the liquid sample, which helps to improve the quality of the product. In an embodiment, the reaction device 10 can withstand a sterilization condition of 127° C. and 30 psi. That is, a sterilization process under 127° C. and 30 psi or a sterilization process under a temperature lower than 127° C. and lower than 30 psi can be performed in the reaction device 10.

In an embodiment, the reaction apparatus 1 may include a lifting system 20. The reaction device 10 can be used in conjunction with the lifting system 20. The lifting system 20 includes a support bracket 201, a suspension arm 202, a winding device 203, a rope 204 and a hook 205. The suspension arm 202 is on the support bracket 201. The suspension arm 202 can be rotated with respect to the support bracket 201. The winding device 203 is disposed on the support bracket 201 or on the suspension arm 202. The rope 204 is on the suspension arm 202. An end of the rope 204 may be connected to the winding device 203, and another end of the rope 204 may be connected to the hook 205. The winding device 203 can be used to retract the rope 204 and release the rope 204 to change the height of the hook 205. The hook 205 can be used to removably connect an object to be moved, such as any element or product in the reaction device 10. For example, as shown in FIG. 1, the hook 205 is connected to the cover 131 of the reaction device 10 through a connecting rope, and the cover 131 can be lifted and separated from the first tank 101 by activating the winding device 203; alternatively, the cover 131 can be lifted and disposed on the first tank 101 by activating the winding device 203. For example, the V-shaped handles 302 may be hung on the hook 205 so as to move the second tank 102; he second tank 102 can be moved out of the accommodation space S1 of the first tank 101 or moved into the accommodation space S1 of the first tank 101 by the lifting system 20. The lifting system 20 is not limited to the type described in the present disclosure. Any type of lifting system can be used in the present disclosure. For example, the lifting system may be a crane.

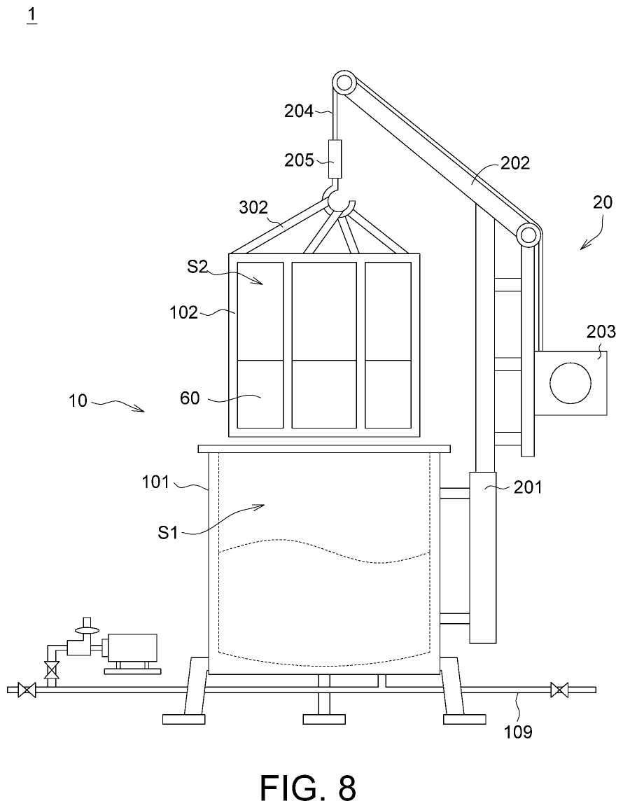

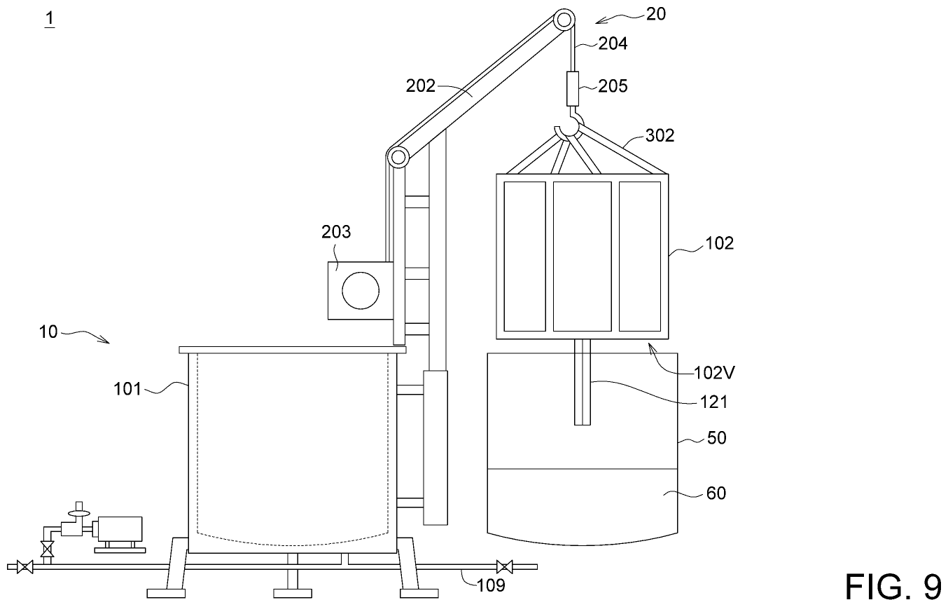

Referring to FIGS. 1, 8 and 9. FIGS. 8-9 illustrate schematic views of operations of the reaction apparatus 1 according to an embodiment of the present disclosure. Some elements in the reaction apparatus 1 are omitted in FIGS. 8-9 for clarity of discussion. The elements in this embodiment which are the same as or similar to those in above embodiments are denoted by the same reference signs; the descriptions related to these element are described above, and will not be repeated here. After the reaction in the reaction device 10 is completed, the cover 131 can be separated from the first tank 101 by the lifting system 20. Then, the V-shaped handles 302 of the second tank 102 of the reaction device 10 are hung on the hook 205 of the lifting system 20, and the second tank 102 is moved upwards until it can move laterally (for example, the second tank 102 is moved upwards until the second tank 102 is completely higher than the first tank 101) by activating the winding device 203, as shown in FIG. 8. Then, the suspension arm 202 supporting the rope 204 can rotate an angle, such as 180 degrees, to move the second tank 102 and the product 60 in the second tank 102 above the storage barrel 50, as shown in FIG. 9. For example, the storage barrel 50 may be a flexible packing material or a hard packing case. Then, the outlet door 121 is moved to a release position, and the product 60 in the second tank 102 is transferred from the second tank 102 to the storage barrel 50 through from the outlet 102V. Processing steps such as sub-packaging, packaging or storage of the product 60 may be performed as required.

In an embodiment, in the process shown in FIGS. 8-9, the pumping device 109 can draw off the liquid in the first tank 101, which can accelerate the separation of the product 60 and the liquid, and the liquid in the first tank 101 can be reused. In an embodiment, before the reaction starts, the second tank 102 is located outside the first tank 101, and the solid material can be put into the second tank 102, and then the second tank 102 and the materials in the second tank 102 are moved to the first tank 101 through the lifting system 20. The tank body 101 is used for subsequent reaction steps for subsequent reaction steps. The reaction apparatus 1 can simplify the preparation before the reaction starts and the product storage steps after the reaction is completed.

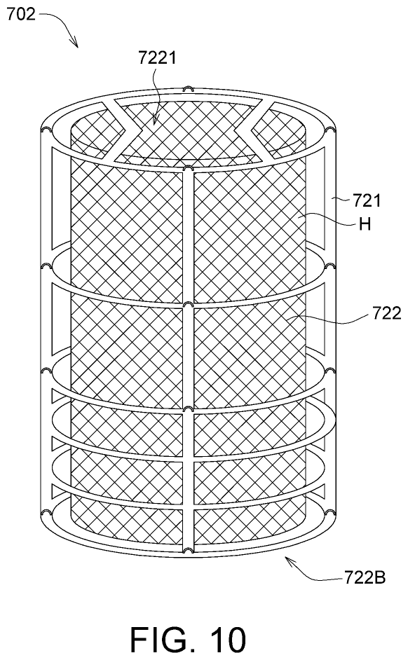

The second tank 102 shown in FIGS. 1-9, for instance, may be a rigid basket (which may be understood as the shape of the second tank 102 is roughly fixed). However, the second tank of the present disclosure is not limited to the type shown in FIGS. 1-9. FIG. 10 illustrates one of the second tanks that can be used in the present disclosure. As shown in FIG. 10, the second tank 702 includes a holder 721 and a filter 722. The filter 722 includes holes H passing through the bottom and/or sidewall of the filter 722.

For example, the filter 722 can be a non-rigid or soft mesh bag, that is, the filter 722 may not have a fixed shape, and its shape can be changed according to the materials contained inside or the way it is arranged on the holder 721. The filter 722 can be removably arranged on the holder 721. The holder 721 is used to support the filter 722 and can improve the rigidity of the second tank 702. The filter 722 can be made of a heat-resistant, acid-resistant and alkali-resistant material, such as filtering cloth, plastic (such as polypropylene (PP), polyethylene (PE), polytetrafluoroethylene (PTFE), etc.) and fiber fabric thereof, or carbon fiber cloth, etc. The solid material (such as biochar) can be disposed in the filter 722. In an embodiment, the filter 722 may be a sieve meeting ASTM E-11 specifications for the number 70 sieve to the number 300sieve. In an embodiment, the filter 722 may be a sieve meeting ASTM E-11 specifications for the number 200 sieve to the number 300 sieve and withstand the temperature above 130° C. The holder 721 can be made of stainless steel or other heat-resistant, acid-resistant and alkali-resistant materials. For example, the holder 721 can be made of SUS304 or SUS316. When the reaction performed in the reaction device is completed, the filter 722 and the solid products (such as microbial-based composite materials on the basis of biochar) in the filter 722 are removed from the holder 721 for storage of the solid products in the filter 722. For example, the filter 722 and the solid products in the filter 722 may be packaged together for storage. The weight of the reaction device can be reduced with the use of the second tank 702, and a lightweight effect can be achieved. Product storage and packaging can be easier with the used of the removable filter 722.

In this embodiment, the filter 722 shown in FIG. 10 includes a supply inlet 7221. The materials used in the reaction can enter the filter 722 from the supply inlet 7221. In the product storage and packaging stage after the reaction is completed, the product can be taken out through the supply inlet 7221. In an embodiment, the filter 722 shown in FIG. 10 may include an outlet and an outlet door at the bottom 722B. The outlet door is movable between a release position and a closed position. When the outlet door is in the release position, the solid products in the filter 722 can be removed from the filter 722 through the outlet. When the outlet door is in the closed position, the outlet door covers the outlet and the outlet is closed. The outlet door may be connected to the bottom 722B of the filter 722 through any type of fastener (e.g. zippers, Hook-and-loop fasteners, snap fasteners, buttons, etc.). In an embodiment, the outlet door is connected to the bottom 722B of the filter 722 through a zipper made of stainless steel or plastic (e.g. polypropylene, polyethylene, Teflon, etc.).

The reaction apparatus 1 of the present disclosure can be used to produce various agricultural bio-based composite materials. One of agricultural bio-based composite materials that can be produced through the reaction apparatus 1 of the present disclosure is illustrated below by taking a microbial-based composite material on the basis of biochar (or it can be understood as using biochar as a host or support) as an example. The cleaned biochar (such as granular biochar or surface modified biochar) is put into the second tank 102 of the reaction device 10. Water (such as water without chlorine or clean water) is added into the first tank 101, and the biochar is immersed in water. A microbial nutrient is added into the first tank 101. The lid assembly 103 is fixed on the first tank 101, and the driving motor 151 is turned on to make the stirrer 152 work. In the process of mixing, acid or basic substance may be added as appropriate to adjust pH value. Then, the temperature and the heating time of the heater 106 are controlled by the temperature controller and the time controller so that gas in the pores of the biochar is replaced with the nutrient liquid or the nutrient liquid can fully contact the surface of the biochar and the inner wall of the pores. A sterilization process under high temperature and high pressure is performed to the materials in the first tank 101 to reduce pollution. After the sterilization process under high temperature and high pressure, cold gas is introduced into the first tank 101 through the intake conduit 182 and the cooling device 184 to lower temperature in the first tank 101. After cooling, the strains to be cultivated are added into the first tank from the inlet 132. The bacteria multiply and are distributed on the surface of the biochar and in the pores after a period of time (such as several days). The extent to which the reaction has proceeded can be known through the sampling pipe 110. For example, a scanning electron microscope (SEM) can be used to observe the distribution of bacteria on the surface and in the pores of biochar. After the embedding is completed, the cover 131 is removed by the lifting system 20, and the second tank 102 is lifted from the first tank 101. The biochar including bacteria which is agglomerated or with larger particles can be separated from the bacteria liquid including suspended biochar. The biochar including bacteria which is agglomerated or with larger particles is a kind of microbial-based composite material on the basis of biochar.

The reaction device according to the present disclosure has the functions and characteristics of a fermentation tank and an autoclave that can provide a high temperature and high pressure environment, and can produce agricultural bio-based composite materials through a one-pot reaction process. That is, a plurality of reaction steps can be performed continuously in the first tank and the second tank without the need to transfer the container during the reaction, which can effectively reduce the pollution of the reaction process, avoid the loss of the container transfer process, simplify the process, save time, increase yield and improve product quality. In addition, the reaction device according to the present disclosure is suitable for mass production of various agricultural bio-based composite materials. Moreover, sterilization processes to the materials (such as biochar) and culture container (such as the first tank and/or the second tank) can be performed at the same place, which can simplify the process. The configuration of the first tank and the second tank can improve the separation effect of solids and liquids, which makes product storage and packaging easier. In addition, microbes can be embedded in the pores of the biochar and microbial-based composite materials on the basis of biochar can be produced with the use of the reaction apparatus. Microbial-based composite materials have the benefits of microbial fertilizers, can be used in soil modification and control of pest, and can ensure the application effect of microbial fertilizers, thereby effectively promoting crop growth and improving crop quality and yield.

It is noted that the elements and methods as described above are provided for illustration. The disclosure is not limited to the configurations and procedures disclosed above. Other embodiments with different configurations of known elements can be applicable, and the exemplified elements could be adjusted and changed based on the actual needs of the practical applications. It is, of course, noted that the configurations of figures are depicted only for demonstration, not for limitation. Thus, it is known by people skilled in the art that the related elements and layers in a semiconductor element, the shapes or positional relationship of the elements and the procedure details could be adjusted or changed according to the actual requirements and/or manufacturing steps of the practical applications.

While the disclosure has been described by way of example and in terms of the exemplary embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A reaction apparatus, comprising:
a first tank having an accommodating space;
a second tank in the accommodating space of the first tank and comprising holes;
a cover on an opening of the first tank;
a stirring assembly on the cover and comprising a driving motor and a stirrer connected to the driving motor, wherein the stirrer is in the accommodating space of the first tank;
a heater in the accommodating space of the first tank;
a controller coupled to the heater to control the heater; and
a gas delivery system on the first tank and in communication with the accommodating space of the first tank.

2. The reaction apparatus according to claim 1, wherein the gas delivery system comprises an intake conduit and an exhaust conduit.

3. The reaction apparatus according to claim 2, wherein the gas delivery system comprises a cooling device connected to the intake conduit.

4. The reaction apparatus according to claim 2, wherein the gas delivery system comprises a vortex cooler connected to the intake conduit.

5. The reaction apparatus according to claim 1, further comprising a buckle, wherein the buckle exerts a force on the cover to push the cover to the first tank and fix the cover on the first tank.

6. The reaction apparatus according to claim 1, further comprising an inlet on the cover and in communication with the accommodating space of the first tank.

7. The reaction apparatus according to claim 1, further comprising a relief valve on the cover and in communication with the accommodating space of the first tank.

8. The reaction apparatus according to claim 1, further comprising a sampling pipe on the first tank and in communication with the accommodating space of the first tank.

9. The reaction apparatus according to claim 1, wherein the controller comprises a temperature controller and a time controller.

10. The reaction apparatus according to claim 1, further comprising a pumping device on the first tank and in communication with the accommodating space of the first tank.

11. The reaction apparatus according to claim 1, wherein the second tank comprises a holder and a filter, and the filter comprises the holes.

12. The reaction apparatus according to claim 1, further comprising a lifting system removably connected to the cover to separate the cover from the first tank.

13. The reaction apparatus according to claim 1, further comprising a lifting system removably connected to the second tank to move the second tank out of the accommodating space of the first tank.
